(19) United States
(12) Patent Application Publication
(10) Pub. No.: US 2009/0220592 A1
(43) Pub. Date: Sep. 3, 2009

(54) PREPARATION FOR THE TREATMENT OF TINNITUS

(76) Inventor: Uwe Stoldt, München (DE)

Correspondence Address: LANDO & ANASTASI, LLP, ONE MAIN STREET, SUITE 1100, CAMBRIDGE, MA, 02142, US

(21) Appl. No.: 12/282,691

(22) PCT Filed: Mar. 15, 2007
(86) PCT No.: PCT/EP2007/002294

§ 371 Date: Feb. 11, 2009

(30) Foreign Application Priority Data
Mar. 15, 2006 (DE) 10 2006 011 922.3

(51) Int. Cl. A61K 36/45; A61K 9/48; A61P 27/16

(52) U.S. Cl. 424/456; 424/732

(57) ABSTRACT

The invention relates to the use of blueberry extract for the preparation of a therapeutic composition for oral administration for the treatment and prevention of tinnitus.

The invention relates to a preparation for oral administration for the treatment of tinnitus which contains blueberry extract as well as antioxidants and micronutrients selected from magnesium oxide, vitamin B1, vitamin B2, vitamin B6, vitamin B12, evening primrose oil and folic acid.

The use of blueberries, as fresh fruit or as (dry) extract for the prevention and treatment of circulatory disorders such as variscose veins, hemorrhoids, diarrhea and a number of ophtalmopathies such as cataract, diabetic retinopathy, glaucoma, macular degeneration and night blindness as well as of excessive strain of the eyes (computer) is widespread.

Tinnitus is a functional disorder of the auditory system which may originate in different levels and structures, without ascertained knowledge as to the pathopsychology of tinnitus being available.

The term “Tinnitus aurium” (Latin: “ear ringing”) or short tinnitus relates to a symptom where the afflicted hears sounds which have no exterior source which would be perceptible to other people. In particular if the disorder is chronic, the psychological strain caused by continuous noise in the ear will be substantial. In the course of the disease, many patients develop secondary symptoms such as sleep disorders, concentration disorders, anxiety neuroses and depressions. In many cases, normal life is no longer possible or only to a limited extent. Patient may be faced with occupational or general disability. Due to this strain, many tinnitus patients are suicidal.

The cause is assumed to be a multifactorial event. Since ascertained knowledge as to the pathopsychological basis of tinnitus is not available, a systematic rational medicinal therapy is not possible. In general, the present therapeutic measures are based on multiple approaches and comprise stress management measures, noise masking with special hearing aids and also surgery. However, these therapeutic approaches show no satisfactory results and there is urgent need for an effective preparation for the treatment of tinnitus.

Surprisingly, it was possible to demonstrate in clinical tests that the preparation of the invention, which contains blueberry extract in combination with specific antioxidants and micronutrients, allowed very significant success in the treatment of tinnitus. Tests with volunteers showed that, after a three-month treatment consisting in daily administration of 2×3 capsules, the tinnitus ear noises were significantly reduced or disappeared.

Thus, the present invention provides a new highly effective and purely natural pharmaceutical or dietary preparation which is suitable for the prevention and treatment of tinnitus/acute hearing loss. Contrary to the preparations which are conventionally used, the pharmaceutical preparations of the invention are natural and purely herbal formulations which are also accepted and well tolerated by patients having a negative attitude towards allopathy.

The blueberry extract of the invention is a dry extract from the berries of Vaccinium myrtillus, i.e. of the European blueberry which, according to Anglosaxon usage, is also called “bilberry”.

A particularly preferred dry extract of the invention is standardized to at least 25% anthocyanosides.

A typical extract used according to the invention contains up to 4.5% water, up to 5% mineral constituents (sulfuric ashes), up to 5% free anthocyanidines and 25-27.5% glycosidically bound anthocyanidines (anthocyanosides). Such extract is commercially available, e.g. as “Bilberry Purified Dry Extract”. The dry extract can be obtained by methods known per se from liquid drug extracts which are prepared by means of percolation, maceration, soxleth method, digestion, particularly, according to pharmacopoeiae instructions (e.g. Bonati A., J. Ethnopharmacol. 1991, April; 32 (1-3): 195-7).

In particular, the blueberry extract used according to the invention can be prepared as briefly described in the following.

Fresh blueberries are pressed to obtain a blueberry juice concentrate which, subsequently, is subjected to ultrafiltration and to ethanol extraction or aqueous alcohol extraction. The extract obtained is concentrated under vacuum to form a paste and dried to form a powder which, then, is ground for obtaining the dry blueberry extract.

An extract of this kind is described in WO 05/092330.

A preferred formulation of the invention contains per dosage unit, such as tablet or capsule:

50 to 500 mg blueberry extract, 10 to 200 mg magnesium (in form of magnesium oxide), 0.2 to 5 mg vitamin B1, 0.2 to 5 mg vitamin B2, 0.2 to 5 mg vitamin B6 and 0.1 to 1 pg vitamin B12.

Further preferred ingredients are folic acid (0.01-10 mg) and evening primrose oil (10-100 mg).

The following formulation is more particularly preferred, in particular, if it is in form of a soft gelatine capsule (amount per dosage unit):

| | | |
|---|---|---|
| blueberry extract | 100-200 | mg |
| magnesium (in form of magnesium oxide) | 10-20 | mg |
| vitamin B1 | 0.2-1 | mg |
| vitamin B2 | 0.2-1 | mg |
| vitamin B6 | 0.2-1 | mg |
| vitamin B12 | 0.1-1 | μg |
| folic acid | 0.01-1 | mg |
| and | | |
| evening primrose oil | 30-60 | mg |

The formulation below is most particularly preferred, in particular, in form of a soft gelatine capsule (amount per dosage unit):

| | | |
|---|---|---|
| blueberry concentrate | 150 | mg |
| evening primrose oil | 50 | mg |
| magnesium (in form of magnesium oxide) | 18.75 | mg |
| folic acid | 0.05 | mg |
| vitamin B1 | 0.312 | mg |
| vitamin B2 | 0.625 | mg |
| vitamin B6 | 0.396 | mg |
| vitamin B12 | 0.225 | μg |

In addition, the dietary or pharmaceutical compositions of the invention may contain pharmaceutical adjuvants such as fillers (carriers), e.g. soy bean oil or partially hydrogenated soybean oil, beeswax, butterfat and lecithin for soft gelatine capsules, disintegrants, binding agents, flow regulation agents, lubricants, emulsifiers, solvents or sorbents.

The dietary or pharmaceutical compositions of the invention can be formulated in any dosage form that is suitable for oral administration according to the standard methods used in the technical field, with solid dosage forms for oral administration such as tablets, hard gelatine capsules and soft gelatine capsules being preferred. Soft gelatine capsules are most particularly preferred. According to the invention, soft gelatine capsules are preferably oval soft gelatine capsules with a size of 2 to 20 minim. and oblong soft gelatine capsules with a size of 6 to 22 minim., most preferably oval soft gelatine capsules with a size of 10 minim.

In this context, it is also preferred that the dosage form is present in specific primary packages such as press-through packages or blister packs, i.e. that it is blistered.

The results described below clearly show that the composition of the invention, which contains blueberry extract in combination with specific antioxidants and micronutrients, allows to achieve very significant therapeutic success in the treatment of tinnitus.

### A. APPLICATION EXAMPLES

### 1. Patient 1, Female, Born in 1948

The patient has been suffering from ear ringing approximately since 2-3 years. She perceives the ear ringing mostly as a whooshing sound on the left side. According to her statement, she did not see a physician in this matter and she did not take any medicaments against this disorder either. The ear ringing did not interfere with her daily routine. Only before falling asleep, she found the ear ringing to be unpleasant. The patient started to take the composition of the invention about eighteen months ago (approximately in the middle of 2005) and takes 2 capsules in the morning and 2 capsules in the evening, not always on a regular basis. In case of migraine bouts, she sticks to the daily intake suggested on the package leaflet.

Result: The ear ringing disappeared six months after she started to take the preparation. However, the patient again perceives the ear ringing in attenuated form if she forgets to take the preparation of the invention. The patient tolerates the composition well. At first, she suffered from indigestions (constipation), which, however, soon ceased.

### 2. Patient 2, Female, Born in 1952

The patient has been suffering from tinnitus since 1993. The tinnitus was diagnosed by an otorhinolaryngologist by means of otoscopy, measurement of hearing threshold and further methods. The patient perceives the tinnitus as a whistling sound in the left ear. She does not take any medicaments against the disorder and she was not given any other therapeutic treatment. The tinnitus does not interfere with the patient's daily routine. She states that the level of the noise is “moderate”.

Since 18 months, the patient has been taking 3 capsules of the composition of the invention in the morning, on a regular basis.

Result: Already 14 days after the patient started to take the composition of the invention, the ear ringing improved. At present, the noise still exists but in attenuated form. The feeling of pressure in the ear associated with the tinnitus has completely disappeared. The patient tolerates the composition very well.

### 3. Patient 3, Female, Born in 1953

The patient has perceived ear ringing in both ears approximately since 1997 and perceives these as a whistling sound. She describes that the ear ringing started subsequent to a vertebral fracture and tooth problems. She had no diagnostic examination relating to tinnitus. The patient had no tinnitus therapy other than a moderate music therapy. The ear ringing was described as loud and thus disturbing. The ear ringing partially interfered with the patient's daily routine. Particularly during the night, she found the ear ringing which disturbed her sleep very upsetting. The ear ringing also caused the patient to be more irritable with her family.

The patient started taking the composition of the invention at the beginning of April 2006. At first she took 4 capsules in the morning and 4 capsules in the evening, she then changed the regimen to 3 capsules at a time.

Result: The patient reports that the tinnitus has completely disappeared since the middle of 2006. First, under the initial dose of 4 capsules both in the morning and in the evening, the tinnitus improved on one side, subsequently, it also disappeared in the other ear when she took 3 capsules of the composition of the invention both in the morning and in the evening.

### 4. Patient 4, Female, Born in 1963

The patient has been suffering from tinnitus caused by acute hearing loss since November 2005. The tinnitus was diagnosed by a alternative practitioner. The patient perceived the ear ringing as a loud whistling and whooshing sound mostly in her right ear. During daytime, the noise did not disturb the patient, while she felt extremely disturbed by the noise in the night, the more so as she was not able to relax and to fall asleep. Therapeutic measures, such as acupuncture and massage and the administration of globules adjusted to tinnitus, were not successful.

The patient started taking the composition of the invention in July 2006, since September, she has been taking 3 capsules in the morning and 3 capsules in the evening, on a regular basis. Since October 2006, she has changed the regimen to 2 capsules in the morning and 2 capsules in the evening.

Result: First, the ear ringing gradually lowered and, since the beginning of 2007, it has totally disappeared.

### B. SUMMARY

Subsequent to the administration of the composition of the invention, the ear ringing which, in some cases, had persisted over years completely disappeared in 3 of 4 patients. The ear ringing of one patient considerably improved and the feeling of pressure in her ear disappeared. All patients confirmed that the disappearance and the improvement of the ear ringing clearly are the result of the administration of the composition of the invention. Three patients tolerate the composition of the invention very well, one patient had indigestions when she started to take the composition which, however, ceased.

1. Use of blueberry extract for the preparation of a therapeutic composition for the treatment and/or prevention of tinnitus.

2. The use of claim 1, wherein the blueberry extract is obtained from Vaccinium myrtillus.

3. The use of claim 1 or 2, wherein the blueberry extract is a dry extract with a content of at least 25% anthocyanosides.

4. The use of claims 1 to 3, wherein the composition contains:
blueberry extract as well as antioxidants and micronutrients selected from magnesium (in the form of magnesium oxide) vitamin B2, vitamin B6, vitamin B12, folic acid and evening primrose oil and optionally pharmaceutically acceptable carriers and further adjuvants.

5. The use of claims 1 to 4, wherein a dosage unit contains:

| | |
|---|---|
| blueberry extract | 5-500 mg |
| magnesium (as MgO) | 10-200 mg. |

6. The use of claims 1 to 4, wherein a dosage unit contains:

| | | |
|---|---|---|
| blueberry extract | 50-500 | mg |
| magnesium (as MgO) | 10-200 | mg |
| vitamin B1 | 0-5 | mg |
| vitamin B2 | 0-5 | mg |
| vitamin B6 | 0-5 | mg |
| vitamin B12 | 0-1 | μg |
| evening primrose oil | 0-100 | mg and |
| folic acid | 0-10 | mg. |

7. The use of claims 1 to 4, wherein a dosage unit contains:

| | | |
|---|---|---|
| blueberry extract | 50-500 | mg, |
| magnesium (as MgO) | 10-200 | mg |
| vitamin B1 | 0.2-5 | mg |
| Vitamin B2 | 0.2-5 | mg |
| Vitamin B6 | 0.2-5 | mg |
| Vitamin B12 | 0.1-1 | μg |
| evening primrose oil | 10-100 | mg and |
| folic acid | 0.01-10 | mg. |

8. The use of any one of claims 1 to 7, wherein a dosage unit contains:

| | | |
|---|---|---|
| blueberry extract | 100-200 | mg, |
| magnesium (as MgO) | 10-20 | mg |
| vitamin B1 | 0.2-1 | mg |
| vitamin B2 | 0.2-1 | mg |
| vitamin B6 | 0.2-1 | mg |
| vitamin B12 | 0.1-1 | μg |
| evening primrose oil | 30-60 | mg and |
| folic acid | 0.01-1 | mg. |

9. The use of claims 1 to 8, wherein a dosage unit contains:

| | | |
|---|---|---|
| blueberry concentrate | 150 | mg |
| evening primrose oil | 50 | mg |
| magnesium (as magnesium oxide) | 18.75 | mg |
| folic acid | 0.05 | mg |
| vitamin B1 | 0.312 | mg |
| vitamin B2 | 0.625 | mg |
| vitamin B6 | 0.396 | mg |
| vitamin B12 | 0.225 | μg. |

10. The use of any one of the preceding claims, wherein the dosage unit is in form of tablets, hard or soft gelatine capsules.

11. The use of any one of the preceding claims, wherein the dosage units are provided in blister packs.

12. The use of any one of the preceding claims, wherein the composition is in form of oval soft gelatine capsules of the size of 2 minim. to 20 minim. or in oblong soft gelatine capsules of the size of 6 minim. to 22 minim.

13. The use of any one of the preceding claims, wherein the composition is in form of oval soft gelatine capsules of the size of 10 minim.
